(19) United States
(12) Patent Application Publication
(10) Pub. No.: US 2020/0367723 A1
(43) Pub. Date: Nov. 26, 2020

(54) OPERATION SECTION, ENDOSCOPE INCLUDING OPERATION SECTION, AND TREATMENT INSTRUMENT INCLUDING OPERATION SECTION

(71) Applicant: OLYMPUS CORPORATION, Tokyo (JP)

(72) Inventors: Takuto YOSHINAGA, Tokyo (JP); Tsukasa OTA, Tokyo (JP); Xiongwei WANG, Tokyo (JP)

(73) Assignee: OLYMPUS CORPORATION, Tokyo (JP)

(21) Appl. No.: 16/906,138

(22) Filed: Jun. 19, 2020

Related U.S. Application Data
(63) Continuation of application No. PCT/JP2018/039107, filed on Oct. 19, 2018.

(30) Foreign Application Priority Data
Dec. 19, 2017 (JP) 2017-242987

(51) Int. Cl. A61B 1/005; A61B 1/008; A61B 1/06

(52) U.S. Cl. CPC A61B 1/0051; A61B 1/06; A61B 1/008

(57) ABSTRACT

An operation section includes a pressing member, a stopper, a lever fixing member and, an exterior member. The lever fixing member is positioned with respect to the exterior member such that a projecting portion is continuously brought into contact with the exterior member when the lever fixing member is rotated, and a recessed portion which changes a contact state of the projecting portion with respect to the exterior member along with the rotation of the lever fixing member is formed in the exterior member.

### CROSS REFERENCE TO RELATED APPLICATION

This application is a continuation application of PCT/JP2018/039107 filed on Oct. 19, 2018 and claims benefit of Japanese Application No. 2017-242987 filed in Japan on Dec. 19, 2017, the entire contents of which are incorporated herein by this reference.

### BACKGROUND OF THE INVENTION

### 1. Field of the Invention

The present invention relates to an operation section which includes a shaft which turnably supports an operation member, and a pulley which is mounted on the shaft and tows a towing member by rotating together with the shaft, an endoscope including the operation section, and a treatment instrument including the operation section.

### 2. Description of the Related Art

Recently, an insertion apparatus, for example, an endoscope has been popularly used in a medical field and an industrial field.

The configuration where a bending portion which is bendable in a plurality of directions is disposed in an insertion section of an endoscope is well-known.

The bending portion enhances advancing property of the insertion section at a bending portion in a conduit and, further, the bending portion changes an observation direction of an observation optical system disposed at a distal end portion which is positioned on a more distal end side than the bending portion in the insertion section.

Usually, the bending portion which is provided in the insertion section of the endoscope is formed by connecting a plurality of bending pieces in an insertion direction of the insertion section. With such a configuration, the bending portion is bendable in four directions, that is, in upward, downward, leftward, and rightward directions, for example.

The bending portion is configured to be bendable in any one of the upward, the downward, the leftward, and the rightward directions. In other words, wires which are four towing members are disposed in the bending portion, and distal ends of these wires are fixed to the bending piece positioned on the most distal end side among the bending pieces and are inserted into the insertion section. With such a configuration, when a towing operation is applied to any one of four wires by a bending operation knob which is an operation member disposed in the operation section, the bending portion is bent in any one of the above-mentioned directions.

More specifically, in the bending portion, when a bending operation knob for leftward and rightward bending which is mounted on the operation section is rotatably operated, a pulley for leftward and rightward bending which is mounted on the operation section is rotated by way of a leftward and rightward rotary shaft. Next, either one of a left side chain portion and a right side chain portion of a leftward and rightward bending chain which is a towing member wound around the pulley is towed. As a result, the bending portion is bent either in the leftward direction or in the rightward direction due to towing of the left side wire or the right side wire.

Further, in the bending portion, when a bending operation knob for upward and downward bending which is mounted on the operation section is rotatably operated, a pulley for upward and downward bending which is mounted on the operation section is rotated by way of an upward and downward rotary shaft which is coaxially disposed with the leftward and rightward rotary shaft. Next, either one of an upper side chain portion and a lower side chain portion of the upward and downward bending chain which is a towing member wound around the pulley is towed. As a result, the bending portion is bent either in the upward direction or in the downward direction due to towing of the upper side wire or the lower side wire.

A configuration is also well-known where a left side wire or a right side wire is directly wound around a pulley for leftward and rightward bending, and an upper side wire or a lower side wire is directly wound around a pulley for upward and downward bending.

Further, the bending operation knob for leftward and rightward bending is positioned in an overlapping manner with the bending operation knob for upward and downward bending in an extending direction of the above-mentioned leftward and rightward rotary shaft and upward and downward rotary shaft outside the operation section, and the bending operation knob for leftward and rightward bending is positioned on a remoter side (hereinafter referred to as an upper side) in the extending direction from an exterior member of the operation section with respect to the bending operation knob for upward and downward bending.

Hereinafter, a side where the bending operation knob for upward and downward bending is disposed is referred to as a lower side with respect to the bending operation knob for leftward and rightward bending in the extending direction.

A configuration is also well-known where an operation section includes a lock lever which fixes a bending angle of a bending portion bent in a leftward direction or a rightward direction due to a rotary operation of a bending operation knob for leftward and rightward bending and a bending angle of the bending portion bent in the upward direction or the downward direction by a rotary operation of a bending operation knob for upward and downward bending. Such a configuration is disclosed in U.S. Pat. No. 8,608,649, for example.

U.S. Pat. No. 8,608,649 discloses the configuration of a lock mechanism which includes: a lever fixing member being a second member which is disposed coaxially with a leftward and rightward rotary shaft and an upward and downward rotary shaft, and rotates along with the rotation of a lock lever; a pressing member being a first member which is disposed coaxially with the leftward and rightward rotary shaft and the upward and downward rotary shaft, and is movable upward and downward in an extending direction along with the rotation of the lever fixing member; an O-shaped ring being a leftward and rightward elastic member which is disposed between the pulley for leftward and rightward bending and the pressing member; and an O-shaped ring which is an upward and downward elastic member disposed between the pulley for upward and downward bending and an inner surface of an exterior member being a third member of the operation section.

In the configuration of the lock mechanism disclosed in U.S. Pat. No. 8,608,649, along with the rotation of the lock lever, the lever fixing member is rotated so that the pressing member is moved downward. As a result, the leftward and rightward O-shaped ring is collapsed between the pressing member and the pulley for leftward and rightward bending, and the upward and downward O-shaped ring is collapsed between the inner surface of the exterior member and the pulley for upward and downward bending which is pressed downward by the pulley for leftward and rightward bending which is pressed downward by the pressing member. Accordingly, a braking force is applied to the pulley for leftward and rightward bending and the pulley for upward and downward bending by the respective O-shaped rings.

In the lock mechanism disclosed in U.S. Pat. No. 8,608,649, three inclined surfaces are formed on respective contact surfaces between the lever fixing member and the pressing member in the circumferential direction about respective rotary shafts.

When the braking force is not applied to the respective pulleys, respective crest sides of three inclined surfaces of the lever fixing member and respective valley sides of three inclined surfaces of the pressing member are brought into contact with each other. On the other hand, when a braking force is applied to the respective pulleys, along with the rotation of the lever fixing member, the respective crest sides of the lever fixing member move from the respective valley sides to the respective crest sides while sliding on the respective inclined surfaces of the pressing member so that the pressing member is moved downward.

In U.S. Pat. No. 8,608,649, the lock lever is positioned between the bending operation knob for upward and downward bending and the exterior member of the operation section in the extending direction outside the operation section.

The configuration is adopted where at the position where a braking force is applied to the respective pulleys, a portion of the lock lever falls in a recessed portion formed on an outer surface of the exterior member along with the rotation of the pulley and hence, click sensing is generated so as to notify an operator of applying of a braking force to the respective pulleys.

### SUMMARY OF THE INVENTION

According to an aspect of the present invention, there is provided an operation section which includes: a first member disposed about a shaft, the first member being movable in a direction of the shaft; a stopper configured to prevent rotation of the first member around the shaft by being brought into contact with a portion of the first member; a second member configured to press the first member along with rotation of the second member around the shaft; and a third member to which the stopper is fixed, wherein the second member is positioned with respect to the third member such that a portion of the second member around the shaft is continuously brought into contact with the third member during the rotation of the second member, and a step which changes a contact state of the portion of the second member with respect to the third member along with the rotation of the second member is formed on at least one of the second member and the third member.

According to another aspect of the present invention, there is provided an endoscope including an operation section which includes the above-mentioned operation section.

According to yet another aspect of the present invention, there is provided a treatment instrument including an operation section which includes the above-mentioned operation section.

### BRIEF DESCRIPTION OF THE DRAWINGS

FIG. 1 is a view showing an external appearance of an endoscope which includes an operation section according to an embodiment;

FIG. 2 is a partial cross-sectional view showing a configuration of a bending operation device mounted on the operation section of the endoscope shown in FIG. 1;

FIG. 3 is a perspective view showing a second member shown in FIG. 2 in an enlarged manner and in a reversed manner;

FIG. 4 is a perspective view showing a first member shown in FIG. 2 in an enlarged manner;

FIG. 5 is a partial enlarged exploded perspective view showing a portion surrounded by a V line in FIG. 2 in a reversed manner;

FIG. 6 is a partial enlarged perspective view schematically showing a state where the rotation of the first member in a circumferential direction around a rotary shaft is restricted by a stopper mounted on a third member of the operation section shown in FIG. 2;

FIG. 7 is a partial enlarged perspective view schematically showing a state where the rotation of a thrust plate shown in FIG. 2 in a circumferential direction around the rotary shaft is restricted by a stopper shown in FIG. 6; and

FIG. 8 is a partial enlarged perspective view schematically showing a state where a rotation range of a pulley shown in FIG. 2 in a circumferential direction around the rotary shaft is defined by the stopper shown in FIG. 6.

### DETAILED DESCRIPTION OF THE PREFERRED EMBODIMENT

Hereinafter, an embodiment of the present invention is described with reference to drawings. Drawings are schematic views. Accordingly, it must be noted that a relationship between a thickness and a width of each portion, a ratio between thicknesses of respective portions and the like differ from the corresponding relationships of portions of an actual operation section. Needless to say, portions of the operation section are described with different size relationship or different ratios between the drawings.

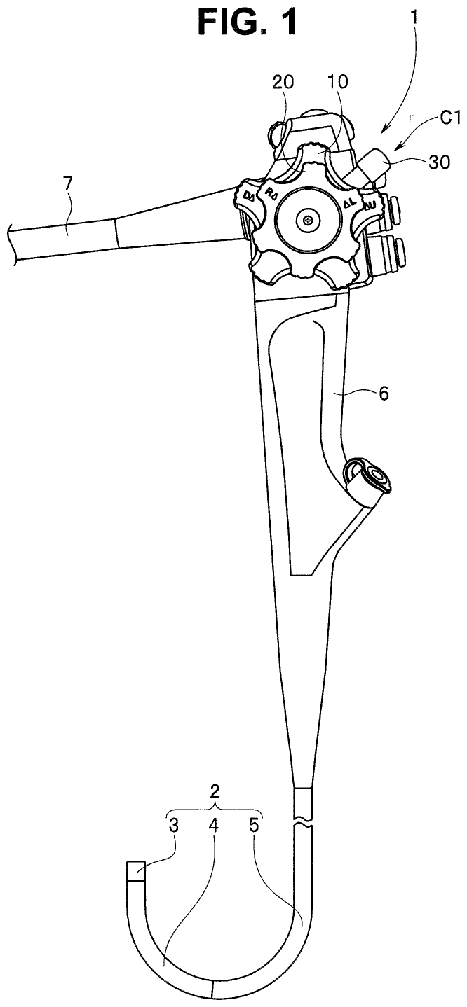

FIG. 1 is a view showing an external appearance of an endoscope which includes an operation section according to the embodiment.

As shown in FIG. 1, a main part of the endoscope 1 is formed of: an insertion section 2 which is inserted into a subject; an operation section 6 which is continuously connected to a proximal end side of the insertion section 2; a universal cord 7 which is extended from the operation section 6; and a connector (not shown) which is mounted on an extension end of the universal cord 7. The endoscope 1 is electrically connected to external devices such as a control device and an illumination device by way of connectors.

On the operation section 6, a bending operation knob 10 for upward and downward bending which is an operation member for bending a bending portion 4 (described later) of the insertion section 2 in upward and downward directions (hereinafter simply referred to as a bending operation knob 10), and a bending operation knob 20 for leftward and rightward bending which bends the bending portion 4 in leftward and rightward directions (hereinafter simply referred to as bending operation knob 20) are mounted.

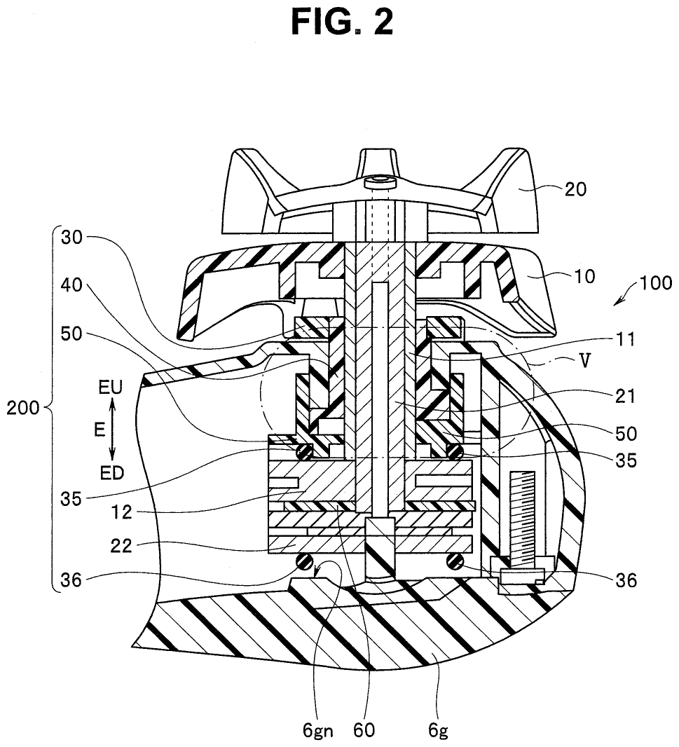

Further, the operation section 6 includes a lock lever 30 which forms a lock mechanism 200 (see FIG. 2) described later which fixes rotary positions of the bending operation knobs 10, 20.

The bending operation knobs 10, 20 and the lock lever 30 form a bending operation device 100 (see FIG. 2) together with other members disposed in the operation section 6.

The insertion section 2 is formed of a distal end portion 3, the bending portion 4, and a flexible tube portion 5, and is formed in an elongated manner in an insertion direction.

In the distal end portion 3, an image pickup unit (not shown) which observes the inside of the subject, an illumination unit which illuminates the inside of the subject and the like are disposed.

The bending portion 4 is bent in four directions, that is, in the upward, the downward, the leftward, and the rightward directions, for example, by a rotary operation of the bending operation knobs 10, 20 by way of the bending operation device 100. With such an operation, the bending portion 4 can change an observation direction of the image pickup unit disposed in the distal end portion 3, and can enhance insertability of the distal end portion 3 in the subject. The flexible tube portion 5 is continuously connected to a proximal end side of the bending portion 4.

Next, the configuration of the lock mechanism 200 in the bending operation device 100 of the endoscope mounted on the operation section 6 is described with reference to FIG. 2 to FIG. 8.

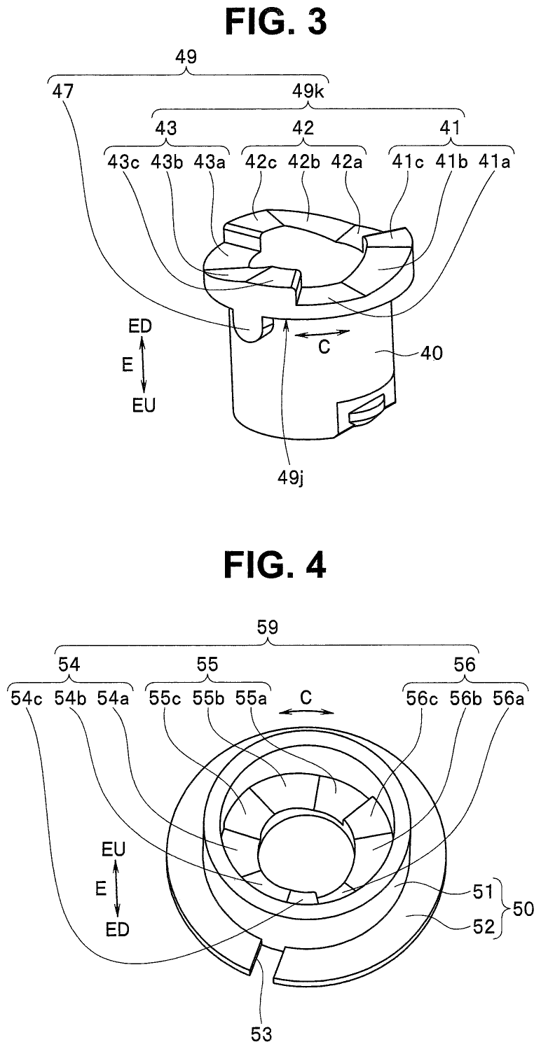

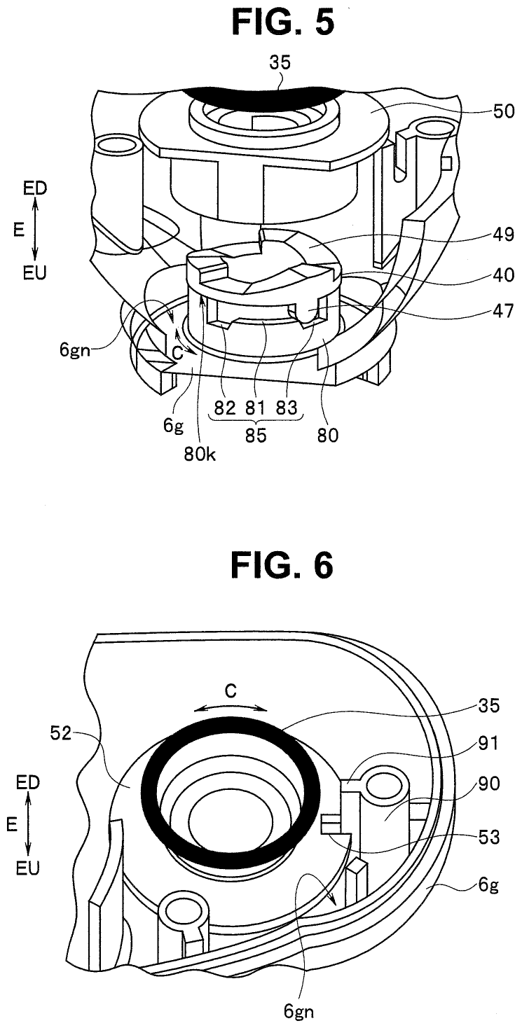

FIG. 2 is a partial cross-sectional view showing a configuration of the bending operation device mounted on the operation section of the endoscope shown in FIG. 1, FIG. 3 is a perspective view showing a second member shown in FIG. 2 in an enlarged manner and in a reversed manner, FIG. 4 is a perspective view showing a first member shown in FIG. 2 in an enlarged manner, and FIG. 5 is a partial enlarged exploded perspective view showing a portion surrounded by a V line in FIG. 2 in a reversed manner.

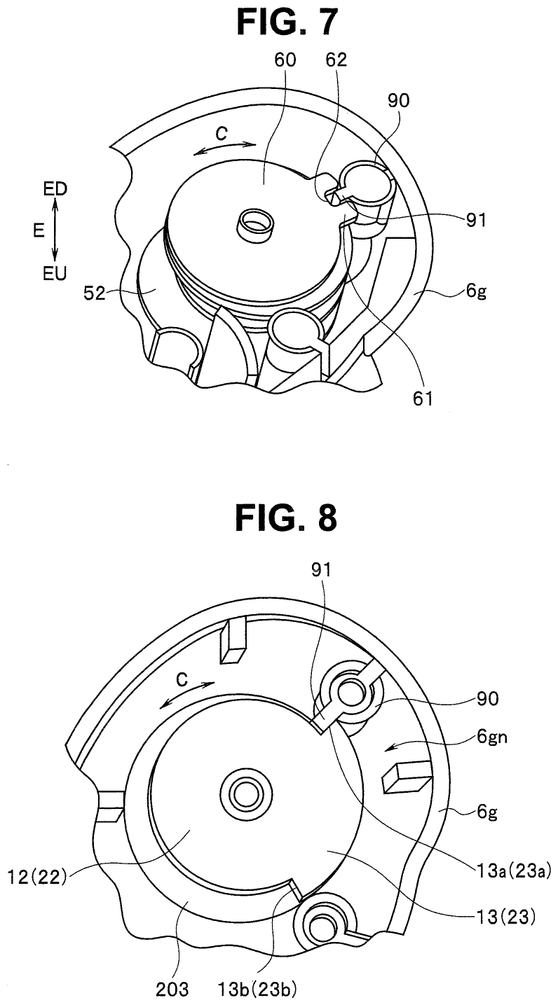

FIG. 6 is a partial enlarged perspective view schematically showing a state where the rotation of the first member in a circumferential direction around a rotary shaft is restricted by a stopper mounted on a third member of the operation section shown in FIG. 2, FIG. 7 is a partial enlarged perspective view schematically showing a state where the rotation of a thrust plate shown in FIG. 2 in a circumferential direction around the rotary shaft is restricted by the stopper shown in FIG. 6, and FIG. 8 is a partial enlarged perspective view schematically showing a state where a rotation range of a pulley shown in FIG. 2 in a circumferential direction around the rotary shaft is defined by the stopper shown in FIG. 6.

As shown in FIG. 2, a main part of the bending operation device 100 is formed of: the bending operation knob 10; the bending operation knob 20; an upward and downward rotary shaft (hereinafter simply referred to as a rotary shaft) 11; a leftward and rightward rotary shaft (hereinafter simply referred to as a rotary shaft) 21; a pulley for leftward and rightward bending (hereinafter simply referred to as a pulley) 12; a pulley for upward and downward bending (hereinafter simply referred to as a pulley) 22; and the lock mechanism 200.

A main part of the lock mechanism 200 is formed of: the lock lever 30; a pressing member 50 which is the first member; a lever fixing member 40 which is the second member; and O-shaped rings 35, 36 which are elastic members.

The rotary shaft 11 having a cylindrical shape is disposed in an extending manner toward an upper side EU from the inside of the operation section 6 in an axial direction (hereinafter referred to as an extending direction) E of the rotary shaft 11. The bending operation knob 10 is fixed to a top portion of the rotary shaft 11 on the upper side EU. With such a configuration, the bending operation knob 10 is rotatable together with the rotary shaft 11 in one direction or the other direction with respect to a circumferential direction C around the rotary shaft 11. In other words, the rotary shaft 11 rotatably supports the bending operation knob 10.

A fixing structure for fixing the bending operation knob 10 to the top portion of the rotary shaft 11 is well-known and hence, the description of the fixing structure is omitted.

The rotary shaft 21 having a cylindrical shape is disposed in the rotary shaft 11 in an extending manner toward the upper side EU from the inside of the operation section 6 in the extending direction E, and is rotatable separately from the rotary shaft 11. The bending operation knob 20 is fixed to a top portion of the rotary shaft 21 on the upper side EU. With such a configuration, the bending operation knob 20 is rotatable together with the rotary shaft 21 in one direction or the other direction with respect to the circumferential direction C around the rotary shaft. In other words, the rotary shaft 21 rotatably supports the bending operation knob 20.

The fixing structure for fixing the bending operation knob 20 to the top portion of the rotary shaft 21 is well-known and hence, the description of the fixing structure is omitted.

The pulley 12 disposed in the operation section 6 is mounted on a lower end of the rotary shaft 11 on a lower side ED positioned in the operation section 6.

A leftward and rightward bending chain which is a towing member (not shown) which bends the bending portion 4 in the leftward and rightward directions is wound around the pulley 12.

A proximal end of a left side wire a distal end of which is fixed to a distal end piece among a plurality of bending pieces which form the bending portion 4 is connected to a left side chain portion of the leftward and rightward bending chain, and a proximal end of a right side wire a distal end of which is fixed to a distal end piece among a plurality of bending pieces which form the bending portion 4 is connected to a right side chain portion of the leftward and rightward bending chain.

With such a configuration, when the bending operation knob 10 is rotatably operated in either one direction or the other direction, the rotary shaft 11 also rotates in the same direction as the bending operation knob 10, and the pulley 12 also rotates in the same direction and hence, the either side of the chain is towed, whereby either one of the wires is towed. Accordingly, the bending portion 4 is bent in either the leftward or the rightward direction.

As shown in FIG. 8, a large diameter portion 13 is formed on a portion of the pulley 12 in the circumferential direction C around the rotary shaft, and the large diameter portion 13 has respective end portions 13a, 13b in the circumferential direction C around the rotary shaft. A rotation range of the pulley 12 is defined by contacting of the respective end portions 13a, 13b with a protruding portion 91 formed on an outer periphery of a screw boss 90 which is a stopper raised in the extending direction E from an inner surface 6gn of an exterior member 6g which is a third member of the operation section 6.

In other words, with such a configuration, it is possible to prevent excessive towing of the wire caused by the excessive rotation of the pulley 12. A screw which is used in assembling the exterior member 6g which forms the operation section 6, for example, in mating and fixing two split exterior members 6g to each other is threaded into the screw boss 90.

The rotation range of the pulley 12 can be set as desired by adjusting a length of the large diameter portion 13 in the circumferential direction C around the rotary shaft or a size of the protruding portion 91 in the circumferential direction C around the rotary shaft.

In the present embodiment, the protruding portion 91 formed on the outer periphery of the screw boss 90 is exemplified as the stopper. However, the protruding portion 91 may be raised from the inner surface 6gn.

The pulley 22 disposed in the operation section 6 is mounted on a lower end of the rotary shaft 21 on the lower side ED positioned in the operation section 6.

An upward and downward bending chain which is a towing member (not shown) for bending the bending portion 4 in the upward and downward directions is wound around the pulley 22.

A proximal end of an upper side wire whose distal end is fixed to a distal end piece among a plurality of bending pieces which form the bending portion 4 is connected to an upper side chain portion of the upward and downward bending chain, and a proximal end of a lower side wire whose distal end is fixed to a distal end piece among a plurality of bending pieces which form the bending portion 4 is connected to a lower side chain portion of the upward and downward bending chain.

With such a configuration, when the bending operation knob 20 is rotatably operated in either one direction or the other direction, the rotary shaft 21 also rotates in the same direction as the bending operation knob 20, and the pulley 22 also rotates in the same direction and hence, the either side of the chain is towed, whereby either one of the wires is towed. Accordingly, the bending portion 4 is bent in either the upward or the downward direction.

As shown in FIG. 8, a large diameter portion 23 is formed on a portion of the pulley 22 in the circumferential direction C around the rotary shaft, and the large diameter portion 23 has respective end portions 23a, 23b in the circumferential direction C around the rotary shaft. A rotation range of the pulley 22 is defined by contacting of the respective end portions 23a, 23b with the protruding portion 91. In other words, it is possible to prevent the excessive towing of the wire caused by the excessive rotation of the pulley 22.

The rotation range of the pulley 22 can be set as desired by adjusting a length of the large diameter portion 23 in the circumferential direction C around the rotary shaft or a size of the protruding portion 91 in the circumferential direction C around the rotary shaft.

The configuration may be adopted where wires are directly wound around the pulley without using chains.

A thrust plate 60 is disposed between the pulley 12 and the pulley 22 in the extending direction E.

The thrust plate 60 is provided for preventing the transmission of the rotation of the pulley 12 to the pulley 22 and for preventing the transmission of the rotation of the pulley 22 to the pulley 12.

As shown in FIG. 7, the thrust plate 60 is formed in a shape where a protruding portion 61 which protrudes to the outside of the thrust plate 60 in an outer diameter direction is formed on an outer peripheral surface of the thrust plate 60 in the circumferential direction C around the rotary shaft, and a recessed portion 62 which is indented toward the inside in an outer diameter direction is formed in the protruding portion 61.

The protruding portion 91 formed on the outer periphery of the screw boss 90 engages with the recessed portion 62 formed in the thrust plate 60. Accordingly, the rotation of the thrust plate 60 in the circumferential direction C around the rotary shaft can be prevented.

Returning to FIG. 2, the lever fixing member 40 having a cylindrical shape (shown in FIG. 3) is mounted on an outer periphery of the rotary shaft 11 coaxially with the rotary shaft 11.

The lock lever 30 is fixed to a top portion of the lever fixing member 40 on the upper side EU. Note that the lock lever 30 is positioned at a position C1 in FIG. 1 in the circumferential direction C around the rotary shaft outside the operation section 6 and between the exterior member 6g and the bending operation knob 10 in the extending direction E.

With such a configuration, the lever fixing member 40 rotates together with the lock lever 30. Accordingly, the lever fixing member 40 rotates separately from the rotation of the rotary shaft 11.

As shown in FIG. 3, on a lower end of the lever fixing member 40, a contact portion 49 having a ring shape and having a larger diameter than other portions of the lever fixing member 40 is formed. Three inclined surfaces 41 to 43 are formed on a lower end surface 49k of the contact portion 49 on the lower side ED in the circumferential direction C around the rotary shaft. A projecting portion 47 which protrudes toward the upper side EU is formed on a portion of an upper end surface 47j on the upper side EU in the circumferential direction C around the rotary shaft.

The inclined surface 41 is formed of a valley portion 41a, an inclined portion 41b, and a crest portion 41c, the inclined surface 42 is formed of a valley portion 42a, an inclined portion 42b, and a crest portion 42c, and the inclined surface 43 is formed of a valley portion 43a, an inclined portion 43b, and a crest portion 43c.

As shown in FIG. 5, the upper end surface 49j of the contact portion 49 is brought into contact with a holding member 80 which is formed on the inner surface 6gn of the exterior member 6g in a protruding manner toward the lower side ED in a cylindrical shape and forms a portion of the exterior member 6g. The projecting portion 47 is continuously brought into contact with a cutout surface 85 formed in a portion of a lower end surface 80k of the holding member 80 in the circumferential direction C around the rotary shaft.

The cutout surface 85 is formed of: a transverse portion 81; and recessed portions 82, 83 which are steps formed on both end portions of the transverse portion 81 in the circumferential direction C around the rotary shaft.

The assembled position of the lever fixing member 40 in the circumferential direction C around the rotary shaft is defined due to the contacting of the projecting portion 47 with respect to the cutout surface 85. In other words, the position C1 of the lock lever 30 in the circumferential direction C around the rotary shaft is defined.

The projecting portion 47 slidably moves in a state where the projecting portion 47 is continuously brought into contact with the cutout surface 85 from the recessed portion 83 to the recessed portion 82 by way of the transverse portion 81 due to the rotation of the lever fixing member 40 in one direction due to the rotation of the lock lever 30 in one direction. On the other hand, the projecting portion 47 slidably moves in a state where the projecting portion 47 is continuously brought into contact with the cutout surface 85 from the recessed portion 82 to the recessed portion 83 by way of the transverse portion 81 due to the rotation of the lever fixing member 40 in the other direction due to the rotation of the lock lever 30 in the other direction.

When braking is not applied to the pulleys 12, 22 described later, the projecting portion 47 is fitted in the recessed portion 83, and when braking is applied to the pulleys 12, 22, the projecting portion 47 is fitted in the recessed portion 82.

When the projecting portion 47 moves from the recessed portions 82, 83 to the transverse portion 81 and when the projecting portion 47 moves from the transverse portion 81 to the recessed portions 82, 83, click sensing is generated due to change in a contact state.

In other words, click sensing which is generated due to fitting of the projecting portion 47 into the recessed portion 82 notifies an operator who operates the lock lever 30 of applying braking to the pulleys 12, 22, and click sensing which is generated due to fitting of the projecting portion 47 into the recessed portion 83 notifies the operator who operates the lock lever 30 of releasing applying of braking to the pulleys 12, 22.

Click sensing may be generated by a configuration where, contrary to the above-mentioned configuration, a projecting portion formed on a portion of the lower end surface 80k of the holding member 80 in the circumferential direction C around the rotary shaft is brought into contact with the cutout surface formed on the contact portion 49 of the lever fixing member 40.

As shown in FIG. 4, a main part of the pressing member 50 is formed of: a body portion 51 having a cylindrical shape extending in the extending direction E; and a flange portion 52 having a C shape formed on an outer peripheral surface of the body portion 51 at an intermediate position in the extending direction E.

In the body portion 51 of the pressing member 50, a contact portion 59 having a ring shape is disposed at an intermediate position in the extending direction E. Three inclined surfaces 54 to 56 are formed on the contact portion 59 in the circumferential direction C around the rotary shaft.

A width of the contact portion 59 in an outer diameter direction is set substantially equal to a width of the contact portion 49 in the outer diameter direction.

The inclined surface 54 is formed of a valley portion 54a, an inclined portion 54b and a crest portion 54c, the inclined surface 55 is formed of a valley portion 55a, an inclined portion 55b and a crest portion 55c, and the inclined surface 56 is formed of a valley portion 56a, an inclined portion 56b and a crest portion 56c.

The lever fixing member 40 is fitted in the body portion 51 from the contact portion 49 side of the lever fixing member 40. As a result, the contact portion 49 is brought into contact with the contact portion 59. More specifically, the inclined surfaces 41 to 43 are brought into contact with the inclined surfaces 54 to 56.

Further more specifically, when braking is not applied to the pulleys 12, 22 described later, the crest portion 41c is brought into contact with the valley portion 54a, the inclined portion 41b is brought into contact with the inclined portion 54b, and the valley portion 41a is brought into contact with the crest portion 54c.

Further, the crest portion 42c is brought into contact with the valley portion 55a, the inclined portion 42b is brought into contact with the inclined portion 55b, and the valley portion 42a is brought into contact with the crest portion 55c. Still further, the crest portion 43c is brought into contact with the valley portion 56a, the inclined portion 43b is brought into contact with the inclined portion 56b, and the valley portion 43a is brought into contact with the crest portion 56c.

When the lock lever 30 is rotated in a counterclockwise direction which is one direction from such a state, the crest portion 41c which is brought into contact with the valley portion 54a slidably moves so as to be brought into contact with the crest portion Mc by way of the inclined portion 54b. The crest portion 42c which is brought into contact with the valley portion 55a slidably moves so as to be brought into contact with the crest portion 55c by way of the inclined portion 55b, and the crest portion 43c which is brought into contact with the valley portion 56a slidably moves so as to be brought into contact with the crest portion 56c by way of the inclined portion 56b. The projecting portion 47 slidably moves from the recessed portion 83 to the recessed portion 82 by way of the transverse portion 81.

Due to contacting of the crest portion 41c with the crest portion Mc, contacting of the crest portion 42c with the crest portion 55c, and contacting of the crest portion 43c with the crest portion 56c, the pressing member 50 is pressed by the lever fixing member 40 and moves toward the lower side ED.

In such an operation, as shown in FIG. 6, a cutout 53 formed in the flange portion 52 engages with the protruding portion 91 formed on the outer periphery of the screw boss 90 and hence, the pressing member 50 moves toward the lower side ED in a state where the rotation of the pressing member 50 in the circumferential direction C around the rotary shaft is prevented.

As a result, the pressing member 50 presses the pulley 12, the thrust plate 60, and the pulley 22 toward the lower side ED by way of the O-shaped ring 35.

When the lock lever 30 is rotated in the clockwise direction which is the other direction from such a state, the crest portion 41c which is brought into contact with the crest portion 54c slidably moves so as to be brought into contact with the valley portion Ma by way of the inclined portion 54b. The crest portion 42c which is brought into contact with the crest portion 55c slidably moves so as to be brought into contact with the valley portion 55a by way of the inclined portion 55b, and the crest portion 43c which is brought into contact with the crest portion 56c slidably moves so as to be brought into contact with the valley portion 56a by way of the inclined portion 56b. Further, the projecting portion 47 slidably moves from the recessed portion 82 to the recessed portion 83 by way of the transverse portion 81.

As a result, the pressing member 50, due to the engagement of the protruding portion 91 with the cutout 53, moves toward the upper side EU in a state where the rotation of the pressing member 50 in the circumferential direction C around the rotary shaft is prevented.

Returning to FIG. 2, the O-shaped ring 35 is disposed in contact with the pulley 12 in a state where the O-shaped ring 35 is sandwiched between the pressing member 50 and the pulley 12 in the extending direction E.

The O-shaped ring 36 is disposed in contact with the pulley 22 in a state where the O-shaped ring 36 is sandwiched between the pulley 22 and an inner surface 6gn of the exterior member 6g which the pulley 22 faces, in the extending direction E.

As described previously, when the lock lever 30 is rotated in one direction so that the pressing member 50 is pressed toward the lower side ED by the lever fixing member 40, the O-shaped ring 35 is brought into contact with the pressing member 50 and is collapsed between the pressing member 50 and the pulley 12 whereby a braking force is applied to the pulley 12.

As descried previously, the releasing applying of a braking force to the pulley 12 is performed in such a manner that when the lock lever 30 is rotated in the other direction, the pressing member 50 moves toward the upper side EU and hence, a collapsed state of the O-shaped ring 35 is released.

As descried previously, when the lock lever 30 is rotated in one direction so that the pressing member 50 is pressed toward the lower side ED by the lever fixing member 40, the pulley 12, the thrust plate 60, and the pulley 22 are pressed toward the lower side ED, and the pulley 22 is brought into contact with the inner surface 6gn and hence, the O-shaped ring 36 is collapsed between the pulley 22 and the inner surface 6gn whereby a braking force is applied to the pulley 22.

As described previously, the releasing applying of a braking force to the pulley 22 is performed in such a manner that when the lock lever 30 is rotated in the other direction, the pressing member 50, the pulley 22, and the thrust plate 60 move toward the upper side EU and hence, a collapsed state of the O-shaped ring 36 is released.

In this manner, the lock mechanism 200 described in the present embodiment is configured to apply a braking force simultaneously to the pulleys 12, 22 using one lock lever 30.

The configuration of such a lock mechanism is simplified compared to a lock mechanism which applies a braking force to the pulleys 12, 22 individually and hence, the number of parts can be reduced. Accordingly, mass productivity can be enhanced and a manufacturing cost can be reduced and hence, such a mechanism is greatly useful in the configuration of a disposable endoscope, for example.

Other configurations of the lock mechanism 200 are substantially equal to the corresponding configurations of a conventional lock mechanism.

In this manner, in the present embodiment, the projecting portion 47 which projects toward the upper side EU is formed on a portion of the upper end surface 49j of the contact portion 49 formed on the lower end of the lever fixing member 40 in the circumferential direction C around the rotary shaft. Further, the projecting portion 47 is described with the configuration where the projecting portion 47 is brought into contact with the cutout surface 85 which is formed on a portion of the lower end surface 80k of the holding member 80 in the circumferential direction C around the rotary shaft, and has the transverse portion 81 and the recessed portions 82, 83.

With such a configuration, an operator who assembles the lock mechanism 200 can arrange the lock lever 30 which is fixed to the lever fixing member 40 at the prescribed position C1 in the circumferential direction C around the rotary shaft by assembling the lever fixing member 40 at the position in the circumferential direction C around the rotary shaft with respect to the pressing member 50 such that the projecting portion 47 is brought into contact with the cutout surface 85, that is, by bringing the inclined surface 41 into contact with the inclined surface 54, bringing the inclined surface 42 into contact with the inclined surface 55, and bringing the inclined surface 43 into contact with the inclined surface 56.

Further, the embodiment has been described where the projecting portion 47 slidably moves in a state where the projecting portion 47 is brought into contact with the cutout surface 85 from the recessed portion 83 to the recessed portion 82 by way of the transverse portion 81 due to the rotation of the lever fixing member 40 in one direction brought about by the rotation of the lock lever 30 in one direction, the projecting portion 47 slidably moves in a state where the projecting portion 47 is brought into contact with the cutout surface 85 from the recessed portion 82 to the recessed portion 83 by way of the transverse portion 81 due to the rotation of the lever fixing member 40 in the other direction brought about by the rotation of the lock lever 30 in the other direction, and when the projecting portion 47 moves from the recessed portions 82, 83 to the transverse portion 81, and when the projecting portion 47 moves from the transverse portion 81 to the recessed portions 82, 83, a contact state of the projecting portion 47 changes so that click sensing is generated.

Further, the embodiment has been described where click sensing brought about by fitting of the projecting portion 47 in the recessed portion 82 notifies an operator who operates the lock lever 30 of applying of braking to the pulleys 12, 22, and click sensing brought about by fitting of the projecting portion 47 into the recessed portion 83 notifies the operator who operates the lock lever 30 of releasing applying of braking to the pulleys 12, 22.

With such a configuration, unlike the prior art where a recessed portion is formed on an outer surface of the exterior member 6g, it is possible to notify an operator of click sensing brought about by an operation of the lock lever 30. Accordingly, the outer surface of the exterior member 6g can be formed smoothly and hence, the external appearance of the operation section 6 can be enhanced and there is no concern that a fine waste or the like remains on the outer surface of the exterior member 6g.

As described above, it is possible to provide the operation section 6 of the endoscope 1 which includes the configuration where erroneous assembling of the lock mechanism 200 can be prevented, and the external appearance of the exterior member 6g can be smoothly formed.

In the present embodiment, the description has been made with respect to the case where the rotation of the thrust plate 60 and the pressing member 50 in the circumferential direction C around the rotary shaft is restricted and a rotation range of the pulleys 12, 22 is defined by the protruding portion 91 formed on the outer periphery of the screw boss 90.

In other words, the restriction of the rotation of the thrust plate 60 and the pressing member 50 in the circumferential direction C around the rotary shaft and defining of the rotation range of the pulleys 12, 22 are performed using one protruding portion 91.

In the prior art, the restriction of the rotation of the thrust plate 60 is performed by fitting a shaft formed on the inner surface 6gn of the exterior member 6g in a hole formed in the thrust plate 60.

Further, in the prior art, defining of a rotation range of the pulley 12 is performed by bringing a protruding portion formed on a surface of the pulley 12 which faces the pressing member 50 and a protruding portion formed on a surface of the pressing member 50 which faces the pulley 12 into contact with each other.

Still further, in the prior art, defining of a rotation range of the pulley 22 is performed by bringing a protruding portion formed on a surface of the pulley 22 which faces the inner surface 6gn and a protruding portion formed on the inner surface 6gn into contact with each other.

In other words, conventionally, the restriction of rotation and defining of a rotation range are separately performed using different members respectively.

However, in the configuration of the prior art, the restriction of rotation of the pressing member 50 is performed by two protruding portions formed on an outer peripheral surface of the pressing member 50 and two recessed portions formed on the inner surface 6gn of the exterior member 6g. Accordingly, there is a possibility that fitting of two protruding portions into two recessed portions is erroneously performed during assembling by 180°. In other words, there is a possibility that the assembling position of the pressing member 50 in the circumferential direction C around the rotary shaft is erroneously selected.

The restrict member used for the restriction of the rotation and the restrict member used for defining the rotation range differ from each other and hence, it is necessary to take into account the respective tolerances.

Further, it is necessary to form the protruding portion on the surface of the pulley 12 which faces the pressing member 50 and the surface of the pulley 22 which faces the inner surface 6gn respectively and hence, there is a possibility that ranges where the O-shaped rings 35, 36 are collapsible in the extending direction E are restricted due to the respective protruding portions.

On the other hand, according to the configuration of the present embodiment, the restriction of rotation is performed by fitting the protruding portion 91 of the screw boss 90 in the recessed portion 62 formed on the outer periphery of the thrust plate 60 and in the cutout 53 of the pressing member 50, and the rotation range is defined by bringing the protruding portion 91 of the screw boss 90 into contact with the respective end portions 13a, 23a, 13b, 23b of the large diameter portions 13, 23 formed on the outer peripheries of the pulleys 12, 22 in the circumferential direction C around the rotary shaft. Accordingly, the above-mentioned erroneous assembling of the pressing member 50 can be prevented, and it is sufficient to take into account the tolerance only with respect to one protruding portion 91 and hence, accuracy in the restriction of the rotation and defining of the rotation range can be increased. Further, there is no possibility that collapsing of the O-shaped rings 35, 36 is obstructed and hence, a strength of a braking force applied to the pulleys 12, 22 can be easily adjusted.

Other advantageous effects acquired by the present embodiment are substantially equal to the corresponding advantageous effects acquired by the prior art.

In the above-mentioned embodiments, the endoscope 1 is exemplified as an example of an insertion apparatus. However, it goes without saying that the insertion apparatus is not limited to the endoscope 1, and the present invention is applicable to operation sections of other insertion apparatuses such as a treatment instrument.

What is claimed is:

1. An operation section comprising:
a first member disposed about a shaft, the first member being movable in a direction of the shaft;
a stopper configured to prevent rotation of the first member around the shaft by being brought into contact with a portion of the first member;
a second member configured to press the first member along with rotation of the second member around the shaft; and
a third member to which the stopper is fixed, wherein
the second member is positioned with respect to the third member such that a portion of the second member around the shaft is continuously brought into contact with the third member during the rotation of the second member, and
a step which changes a contact state of the portion of the second member with respect to the third member along with the rotation of the second member is formed on at least one of the second member and the third member.

2. The operation section according to claim 1, further comprising:
a pulley mounted on the shaft, the pulley towing a towing member by being rotated together with the shaft; and
an elastic member disposed in contact with the pulley, wherein
the first member is arranged so as to sandwich the elastic member between the pulley and the first member.

3. The operation section according to claim 2, wherein the portion of the second member is brought into contact with the step with respect to the third member in pressing the pulley by the first member by way of the elastic member due to movement of the first member toward a side of the pulley in the shaft direction.

4. The operation section according to claim 2, wherein braking is applied to the rotation of the pulley by pressing of the pulley by the first member by way of the elastic member due to movement of the first member toward a side of the pulley in the shaft direction.

5. An endoscope comprising the operation section described in claim 1.

6. A treatment instrument comprising the operation section described in claim 1.
